(19) United States
(12) Patent Application Publication
(10) Pub. No.: US 2021/0252499 A1
(43) Pub. Date: Aug. 19, 2021

(54) DEPOSITING MICRODOTS WITH ANALYTES ON ANALYSIS CHIPS

(71) Applicant: Hewlett-Packard Development Company, L.P., Spring, TX (US)

(72) Inventors: Fausto D'Apuzzo, Palo Alto, CA (US); Trinny Tat, Palo Alto, CA (US); Raghuvir N. Sengupta, Palo Alto, CA (US); Steven Barcelo, Palo Alto, CA (US); Anita Rogacs, San Diego, CA (US)

(73) Assignee: Hewlett-Packard Development Company, L.P., Spring, TX (US)

(21) Appl. No.: 17/054,675

(22) PCT Filed: Oct. 11, 2018
(86) PCT No.: PCT/US2018/055489

§ 371 Date: Nov. 11, 2020

(51) Int. Cl. B01L 3/02

(52) U.S. Cl. CPC B01L 3/0268; B01L 2300/168; B01L 2300/0822; B01L 2200/143

(57) ABSTRACT

In one example, an apparatus includes a surface-enhanced substrate having a microdot deposited onto a surface thereon via a microfluidic ejector. The microdot includes a predetermined concentration of an analyte.

### BACKGROUND

Sensors can be fabricated via colloid aggregation, electrochemically roughened metal surfaces, or nanoimprint lithography, among other techniques. For example, nanoimprint lithography creates patterns by mechanical deformation of imprint resist and subsequent processes. The imprint resist is typically a monomer or polymer formulation that is cured by heat or ultraviolet (UV) light during the imprinting.

### BRIEF DESCRIPTION OF THE DRAWINGS

Various features of the techniques of the present application will become apparent from the following description of examples, given by way of example only, which is made with reference to the accompanying drawings, of which:

FIG. 1 is a side view of an example system for depositing and analyzing microdots on analysis chips;

FIG. 2 is a drawing of another system in which a laser provides co-linear illumination with light returned to the detector, in accordance with examples;

FIG. 3 is a top down view and two exploded side views illustrating microdots deposited on an example sensor having collapsible nanopillars;

FIG. 4A is a diagram illustrating a single dot pattern, in accordance with examples;

FIG. 4B is a diagram illustrating a multidot pattern, in accordance with examples;

FIG. 4C is a diagram illustrating a multiconcentration multidot pattern, in accordance with examples;

FIG. 4D is a diagram illustrating a multianalyte pattern, in accordance with examples;

FIG. 4E is a diagram illustrating a multidot multianalyte pattern, in accordance with examples;

FIG. 4F is a diagram illustrating a multiconcentration multianalyte pattern, in accordance with examples;

FIG. 4G is a diagram illustrating an edge pattern, in accordance with examples;

FIG. 4H is a diagram illustrating a combination pattern, in accordance with examples;

FIG. 5A illustrates a separate region for calibration, in accordance with examples;

FIG. 5B illustrates multiple separate regions for calibration, in accordance with examples;

FIG. 5C illustrates a laser-treatable calibration region, in accordance with examples;

FIG. 5D illustrates a detachable region for calibration, in accordance with examples;

FIG. 6 is a schematic diagram illustrating an example method for generating a calibration curve;

FIG. 7 is a schematic diagram illustrating another example method for generating a calibration curve;

FIG. 8 is a schematic diagram illustrating an example method for performing a sensor performance assay to reject defective sensors;

FIG. 9 is a schematic diagram illustrating an example method for filtering sensors based on an estimated performance; and

FIG. 10 is block diagram of an example controller to generate calibration curves and perform analysis of spectral content, in accordance with examples.

### DETAILED DESCRIPTION

Sensors can be fabricated via colloid aggregation, electrochemically roughened metal surfaces, or nanoimprint lithography, among other techniques. However, sensor-to-sensor variability among the fabricated sensors may make working with these sensors difficult and costly. For example, a significant number of sensors that are fabricated may not meet a threshold of performance. Accordingly, many sensors that are shipped may be later found to be lacking in quality and discarded. In addition, analytes used to test the sensors after shipment may perform differently between sensors because of minor irregularities in fabrication. Thus, it may be difficult to quantify the results of assays performed on the sensors using various analytes.

Described herein are techniques for performing assays on analysis chips using deposited microdots having analytes. As used herein, a microdot refers to a deposit of analyte that covers less than an entire surface of an object to be tested. For example, a microdot may be an area of deposited material that includes an analyte of a dispensed volume of between 20 picoliters (pL) to 100 nanoliters (nL). In some examples, a 20 pL droplet forms a microdot having a diameter of about 50 micrometers. As used herein, an analyte refers to any substance suitable for spectroscopic analysis of analysis chips. The analyte may be a molecule, or mixture of molecules.

The techniques enable analysis chips to be tested prior to shipment, providing calibration curves that enable quantitation of subsequent assays using an analyte. Moreover, the techniques include the use of minimal area and various configurations of microdots on the analysis chips such that the microdots used in generating the calibration curves do not affect the subsequent assays. In various examples, the techniques described herein use less than 10% percent of sensor area for calibration of Surface-Enhanced substrates, less than about 5% of the sensor area, less than about 2% of the sensor area or lower.

The techniques described herein also enable the ability to calibrate sensor performance directly by sampling with a range of analytes that can be targeted in the desired application, thus accounting for effects such as surface binding efficiency. The techniques described herein can be applied to almost any surface-enhanced plasmonic substrate, without introducing additional and complex fabrication steps. The techniques described herein can also further be integrated with automated optical interrogation techniques to perform several measurements on the same substrate. Finally, the techniques improve quantitation by generating calibration curves to be used when performing assays on sensors using particular analytes.

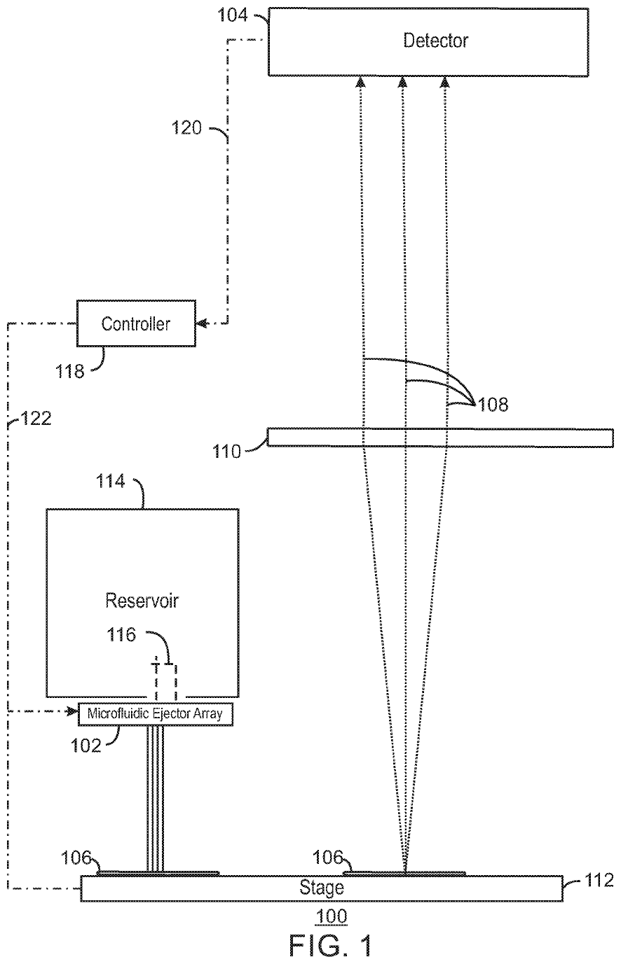

FIG. 1 is a side view of an example system 100 for depositing and analyzing microdots on analysis chips, in accordance with examples. The system 100 has a microfluidic ejector array 102 to deposit microdots and a detector 104 that includes an optical instrument, such as a spectrometer, to collect single point spectra or hyperspectral images of sensors 106 having the microdots thereon. In some examples, the microfluidic ejector array 102 is a thermal inkjet (TIJ) dispense head. The detector 104 may be an imaging system, a multichannel spectrophotometer, or any number of other optical sensors. The detector 104 is used to process the light 108 arriving from one of the sensors 106 and focus the light 108 onto the detector 104.

As described herein, in some examples, the microfluidic ejectors of the microfluidic ejector array 102 use thermal resistors to eject fluid 109 from nozzles by heating to create bubbles that force fluid 109 from the nozzles. In other examples, the microfluidic ejectors use piezoelectric cells to force fluid 109 from the nozzles.

The detector 104 may include lenses, filters, diffraction gratings, and other devices to focus the incoming light 108 on a detector array. In some examples, the detector 104 includes a monochromator that allows a narrow frequency band of the light 108 to reach the detector elements in a spectrometer of the detector 104. In various examples, the monochromator is adjusted to different frequencies of the light 108 for operation. In other examples, the detector 104 divides the incoming light 108 into different channels, each of which are sent to a different sensor within the detector 104, providing multispectral analysis of the incoming light 108. In various examples, the detector 104 are used to perform brightfield, dark-field, florescence, hyperspectral, and other optical analyses. As used herein, a hyperspectral analysis system uses multiple frequencies of light to analyze an image.

A focusing lens 110 is used to focus the light 108 coming from the sensors 106 onto the detector 104. The focusing lens 110 may be a single lens, a group of lenses, or other optical apparatus. In an example, the focusing lens 110 is a Fresnel lens, providing a wide area lens without adding significant complexity. In other examples, the focusing lens 110 is integrated with the optical system, and includes multiple elements, such as a microscopy objective. In some examples, the focusing lens may provide magnification of 4× or greater.

A stage 112 may be moved to place different sensors 106 under the microfluidic ejector array 102, such as individual sensors 106 on a multi-sensor wafer, a set of individual sensors, or any combinations thereof. In some examples, the stage 112 is an x-y-z translation stage, or x-y-z stage, that can move any of a number of sensors 106 in an x-y-z grid in a multi-sensor wafer. In other examples, the stage 112 is a linear translation stage that can move sensors 106 under a microfluidic ejector in the microfluidic ejector array 102 for deposition of microdots onto the surface thereof. The stage 112 may also be used to move different positions of sensors 106 under the MFA 102 to deposit the microdots.

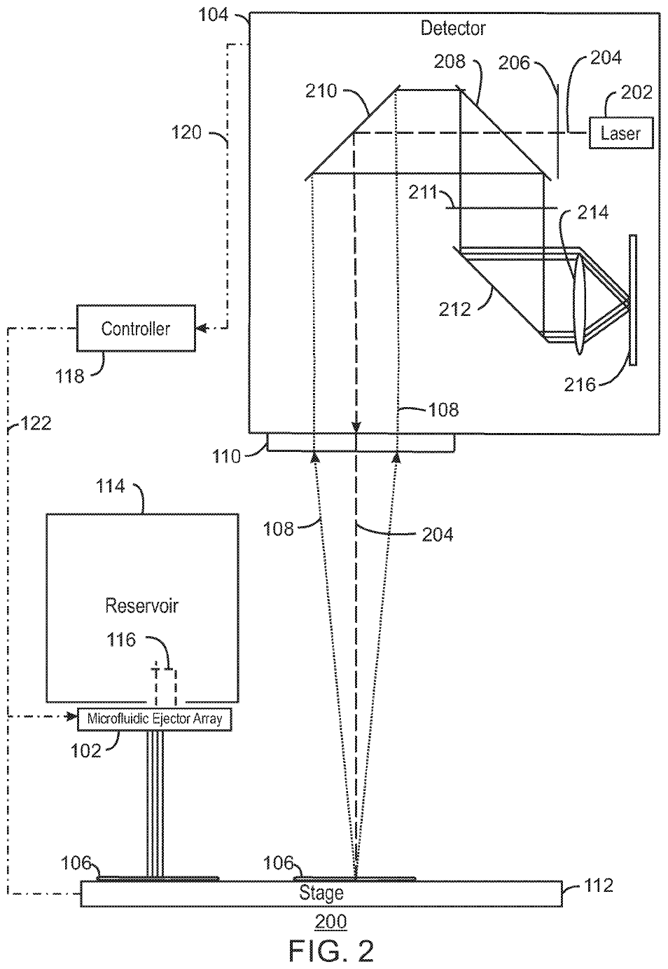

The sensors 106 may be illuminated using any number of different techniques. For example, the detector 104 may include a co-linear illumination system as described with respect to FIG. 2. In some examples, the light source is a laser, such as a laser photodiode.

The reservoir 114 holds a fluid 109 that is to be ejected from the microfluidic ejector array 102. In one example, the fluid 109 includes an analyte. In another example, the reservoir 114 holds a fluid 109 that includes a material of interest, such as molecules, particles, or cells. For example, the material of interest may be an analyte. The reservoir 114 feeds into a chamber 116 that feeds the microfluidic ejector array 102. In one example, the chamber 116 is around 6 mm in size and is fluidically coupled to the nozzles of the microfluidic ejector array 102.

The reservoir 114, chamber 116, microfluidic ejector array 102, stage 112, may form a single material isolation unit. The material isolation unit may be assembled from individual parts, or may be made into a single integrated unit for easier handling.

The system 100 includes a controller 118 that is coupled to the detector 104 through an image data link 120. The controller 118 may analyze images from the camera 104 to identify target emissions, for example, from molecules or particles, molecules, and the like, from microdots in the sensors 106. The controller 118 is also coupled through control links 122 to the microfluidic ejectors of the microfluidic ejector array 102, and to motors controlling the stage 112.

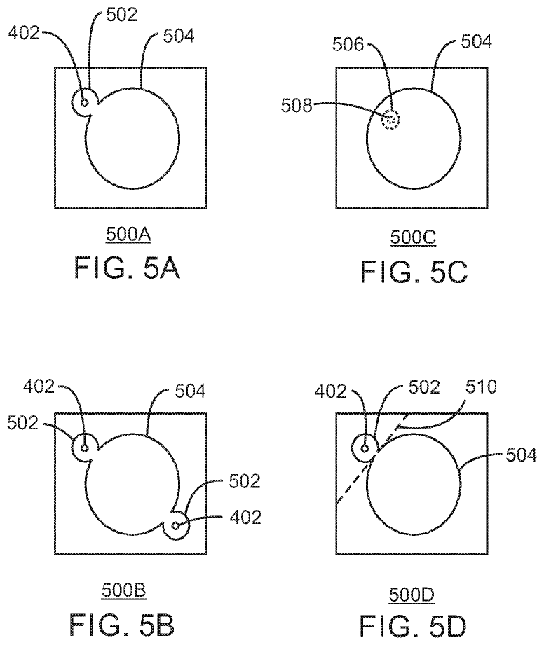

In an example, the controller 118 fires the microfluidic ejectors of the microfluidic ejector array 102. The controller 118 may then cause the stage 112 to be moved to place the sensor 106 under the detector and then cause the detector 104 to analyze of the sensors 106. The stage 112 may be moved to allow depositing onto a calibration region of each of the sensors 106. For example, the calibration region may be a small portion of a surface of the sensors 106, such as a predetermined calibration region reserved for depositing microdots. In some examples, the calibration region may be a separate calibration surface coupled to a side of the sensors 106, that may be detached, as shown and discussed in FIG. 5.

In another example, when the controller 118 detects a target emission from a sensor 106, the controller 118 uses the motors of the stage 112 to move a subsequent sensor 106 into range for analysis by the camera 104. The controller 118 then activates a microfluidic ejector 102 to eject a microdot onto another sensor 106 to be subsequently analyzed. The controller 118 then moves a different sensor 106, to be deposited with microdots and analyzed via the detector 104 in a similar manner.

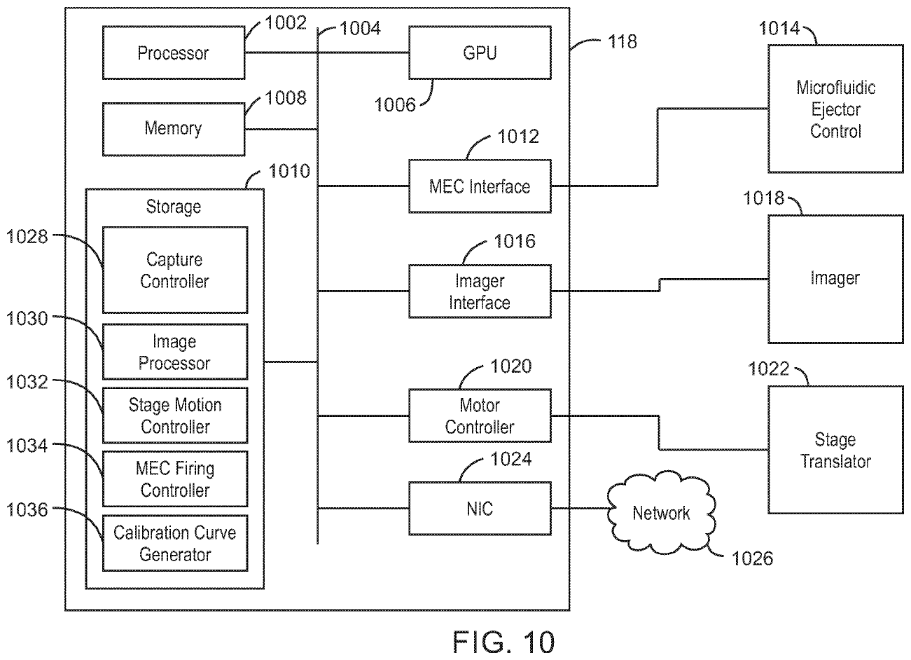

The detector 104 includes an optical device that is used to probe the materials in the microfluidic ejector array 102. In various examples, the optical device is a spectrometer, microscope, fluorimeter, a particle size analyzer, an image recognition system, or a combination thereof. The analysis procedure for the detector 104 is discussed in greater detail with respect to FIG. 2. The controller 118 is discussed in greater detail with respect to FIG. 10.

The block diagram of FIG. 1 is not intended to indicate that the example system 100 is to include all of the components shown in FIG. 1. Further, the system 100 may include any number of additional components not shown in FIG. 1, depending on the details of the specific implementation. For example, the system 100 may include additional sensors 106, light sources, reservoirs 116, detectors 104, etc.

FIG. 2 is a drawing of another system 200 in which a laser 202 provides co-linear illumination with light 108 returned to the detector 104, in accordance with examples. Like numbered items are as described with respect to FIG. 1. In this example, the detector 104 includes a laser 202 that provides a source of illumination 204. The detector 104 further includes a line filter 206, a reflective surface 208, and a reflective source 210. The line filter 206 may be a narrow bandpass filter centered on a particular wavelength. In some examples, the reflective surface 208 is a partially silvered mirror or a prism, or another type of beam splitter, that directs the illumination 204 from the laser 202 through the focusing lens 110 onto the stage 112 to illuminate the sensor 106. In some examples, the laser 202 may alternatively be a co-linear light source that may include any number of sources of illumination. In an example, the co-linear light source 206 includes an array of light emitting diodes. In the example of FIG. 2, the co-linear light source is a laser 202 and optics such as the focusing lens 110. The focusing lens 110 can expand the beam of illumination 204 and direct the beam of incoming light 108 linearly into the optical system 202.

Incoming Light 108 returning from the sensor 106 bounces off reflective surfaces 210 and 208, through edge filter 211, and then bounces of reflective surface 212 to reach a focusing lens 214. The focusing lens 214 focuses the incoming light 108 onto the detector array 216. To enhance the amount of light 108 received by the detector 104, filters may be placed between the laser 202 and the sensors 106 and between the sensors 106 and the detector array 216. In an example, the filters are at an excitation band, such as a 5 nanometer (nm) bandpass filter centered on a wavelength of about 785 nm, at the line filter 206, and at an emission band, such as low-pass edge filter with a cutoff wavelength of about 800 nm, at the edge filter 211. The reflective surface 208 may include a dichroic filter that enables a band of illumination 204 from the laser 202 to pass through, while reflecting incoming light 108. In another example, the filters 208 are polarizing filters that are placed perpendicular to each other.

The block diagram of FIG. 2 is not intended to indicate that the example system 200 is to include all of the components shown in FIG. 2. Further, the system 200 may include any number of additional components not shown in FIG. 2, depending on the details of the specific implementation. For example, the system 200 may include additional sensors 106, light sources 202, reflective optics, filters 206, apertures, reservoirs 114, detectors 104, etc.

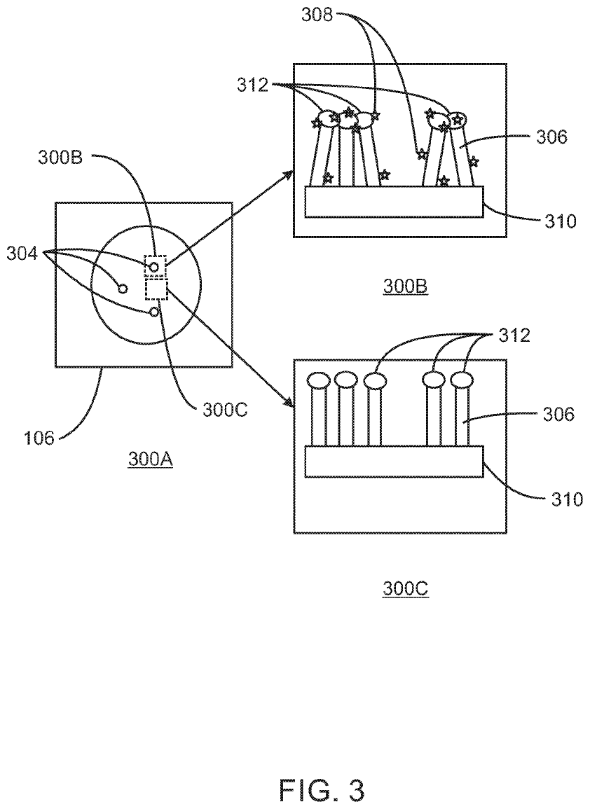

FIG. 3 is a top down view 300A and two exploded side views 300B, 300C illustrating microdots 304 deposited on an example sensor 106 having collapsible nanopillars 306 partially covered with analytes 308 and coupled to a substrate 310. For example, the nanopillars 306 may be polymer shafts with metal caps 312. The collapsible nanopillars 306 may be formed from a column layer on the surface of the substrate 310 by any number processes, including nano-embossing, lithography followed by reactive ion etching or chemical etching, and the like. The column layer may be a polymeric material that can be formed into columns by any number of processes. Polymeric materials that may be used include but are not limited to, photo resists, hard mold resins such as PMMA, soft mold polymers such as PDMS, ETFE or PTFE, or hybrid-mold cross-linked, UV-curable or thermal-curable, polymers based on acrylate, methacrylate, vinyl, epoxy, siloxane, peroxide, urethane or isocyanate. The polymer materials may be modified to improve imprint and mechanical properties with copolymers, additives, fillers, modifiers, photoinitiators, and the like. Any of the materials mentioned with respect to the substrate 310 may also be used. In some examples, the substrate 310 may form a column layer, while in other examples, the collapsible nanopillars 306 may be directly formed on the substrate 310.

In a nano-embossing process, a column layer may be softened and then run through a die to imprint the collapsible nanopillars 306. Any number of other processes known in the art may be used to form the collapsible nanopillars 306 from a column layer. Further, the column layer may be part of the substrate 310 and lithographic and other etching techniques may be used.

In some examples, the collapsible nanopillars 306 may be deposited on the substrate 310, for example, using nano-printing, ion deposition techniques, and the like. In a nano-printing process, the materials forming the collapsible nanopillars 306 may be directly deposited, or printed, on the surface of the substrate 310. In other examples, nano-wires may be grown on the substrate 310 through ion deposition or chemical vapor deposition. In growing the nano-wires to produce the flexible column, nano-wire seeds may be deposited onto the substrate 310. The nano-wire seeds may be silicon nano-structures, and the nano-wires may be silicon dioxide structures grown during chemical vapor deposition from silane. Once the collapsible nanopillars 306 are formed, metal caps may be formed over the nanopillars.

As shown in FIG. 3, the example sensor 106 has three microdots 304 deposited thereon. For example, the three microdots 304 may have been deposited using the system 100 or 200 above. As seen in the first exploded side view 300B, the portions of the sensor 106 with microdots 304 include a number of analyte molecules 308 on and between collapsed nanopillars 306. For example, the analytes may be a type of molecule that has good affinity with metallic substrates. In one example, the analyte is composed of trans-1,2-bis(4-pyridyl)-ethylene (BPE) molecules used with a gold substrate. In some examples, the collapse of the flexible nanopillars is induced by microcapillary forces from an evaporating fluid, such as the ink of the deposited microdots 304. In some examples, a strong enhancement in surface-enhanced luminance may be obtained from the nanopillars when they are collapsed into groups, termed collapsed groups herein. The enhancement is based on intense local electric fields generated by the plasmon resonance of adjacent metal caps at the top of the collapse to nanopillars, which may be separated by a narrow gap on the nanometer (nm) scale.

The nanopillars may be supported by a substrate 310. For example, the substrate 310 may be made from silicon, glass, quartz, silicon nitride, sapphire, aluminum oxide, diamond, diamond-like carbon, or other rigid inorganic materials, such as metals and metallic alloys. In some examples, the substrate 310 may be a polymeric material, such as a polyacrylate, a polyamide, a polyolefin, such as polyethylene, polypropylene, or a cyclic olefin, a polycarbonate, polyesters such as polyethylene terephthalate, polyethylene napthalate, or other polymeric material suitable for making films. Any of these polymeric materials may be a copolymer, a homopolymer, or combination thereof. In some examples, the substrate 310 may be a web used in a roll-to-roll fabrication process. The substrate 310 together with the nanopillars 306 or any other suitable surface enhancement is referred to herein as a surface-enhanced substrate. In some examples, the surface-enhanced substrate is any plasmonic sensing substrate, including nanofabricated substrates, colloidal suspensions on paper, or any other plasmonic enhancement platform. For example, the surface-enhanced substrate may be a Surface-Enhanced Raman Spectroscopy (SERS) surface, a surface-enhanced infrared absorption (SEIRA) surface, or a Surface-Enhanced Luminescence (SEL). Such surface-enhanced substrates may be intrinsically super-hydrophobic because of micro- or nano-pillar or other micro- or nano-structures. The hydrophobic nature of these structures allows calibration droplets to stay localized in a very small area. For example, the area may have a diameter of approximately 50 micrometers for 20 pico-liter droplets.

The microdots 304 can be analyzed via a micro-assay using light reflected off the surface-enhanced substrate to generate calibration curves associated with the sensor 106, as described in greater detail above and below. For example, in response to an excitation beam, electromagnetic radiation may be emitted from the active surfaces in the analysis chips. The characteristics of the emitted radiation may depend, at least in part, on the analyte species, providing information about the analyte species. The metal caps 312 of the collapsed groups provide a plasmon resonance that may interact with the analyte species enhancing the spectroscopic response of the analyte species. In some examples, the excitation beam and the emitted radiation may be at wavelength ranges extending from the near ultraviolet to the near infrared. For example, this may cover a wavelength range from about 150 nanometers (nm) to about 2,500 nm. In some examples, the mid-infrared regions may be included, such as about 3 micrometers (μm) to about 50 μm. Accordingly, analysis chips with sensors 106 having collapsible nanopillars 306 may be used for surface enhanced spectroscopy (SES), such as surface enhanced Raman spectroscopy (SERS), or other surface enhanced luminescence (SEL) techniques, such as fluorimetry or infrared, among others.

In some examples, the microdots 304 are then laser treated to eliminate any residual optical effects from the microdots 304. For example, the analytes 308 in the microdots 304 may be degradable molecules that degrade with laser treatment or any other suitable form of treatment. The sensor 106 may be an analysis chip that can then be tested or analyzed using an analyte. The analysis chip may be tested via an assay by exposing an analyte 308 to the surface of the surface-enhanced substrate. For example, the analysis chips may be dipped into or sprayed by a liquid containing the analytes 308. The resulting analyte-covered analysis chips can be analyzed. The analysis may be aided by the use of the generated calibration curves from the micro-assay analysis. Moreover, the analysis may not be affected by the microdots 304. In case of dynamic substrates, such as collapsible nanopillars 306, the techniques described herein allow to interrogate a small substrate area while leaving most of the sensor region untouched. In some examples, greater than 99% of the total surface-enhanced substrate area may be unaffected by the microdots 304.

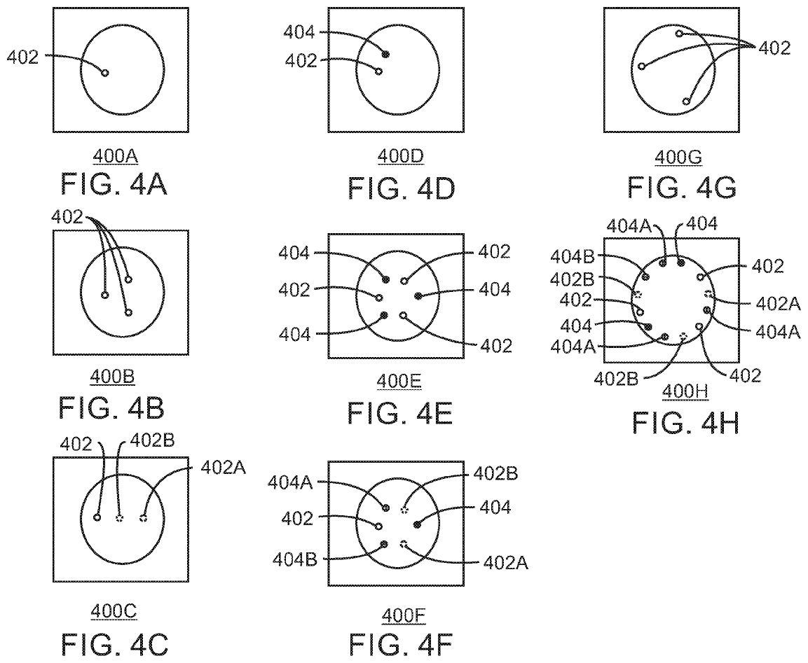

The block diagram of FIG. 3 is not intended to indicate that the example sensor 106 is to include all of the components shown in FIG. 3. Further, the sensor 106 may include any number of additional components not shown in FIG. 3, depending on the details of the specific implementation. For example, the sensor 106 may include additional microdots 304, nanopillars, etc. A variety of microdot patterns that can be used are described with respect to FIG. 4. Furthermore, in some examples, the microdots are located in calibration regions that may be coupled to the sensor 106. For example, the calibration regions may be detached prior to analyzing the chips, as described in FIG. 5. In addition, although examples herein focus on the use of the flexible nanopillars, any number of other flexible columnar structures made using various techniques may be used in the design groups. These may include flexible columnar structures grown as nano-wires, conical structures formed by vapor etching, or any number of other structures.

FIGS. 4A-4H are diagram illustrating various example patterns for depositing microdots onto a sensor. FIG. 4A is a diagram that illustrates a single dot pattern 400A, in accordance with examples. As shown in FIG. 4A, the single dot pattern 400A includes the use of a single microdot 402 having a predetermined amount of a single analyte. For example, each sensor to be analyzed may receive a single microdot 402 during depositing. The use of a single dot pattern 400A may minimize area used for the micro-assay, thus resulting in a larger area available for a subsequent assay.

FIG. 4B is a diagram that illustrates a multidot pattern 400B, in accordance with examples. The multidot pattern 400B of FIG. 4B illustrates the use of multiple microdots 402. For example, the multiple microdots 402 may have the same predetermined concentration of analyte. The multidot pattern 400B may be used to sample multiple points on a surface-enhanced substrate and average the resulting measurements to generate a more accurate calibration curve based on the averaged measurements.

FIG. 4C is a diagram that illustrates a multiconcentration multidot pattern 400C. The multiconcentration multidot pattern 400C of FIG. 4C illustrates the use of multiple concentrations of an analyte in multiple microdots 402A, 402B, 402C across a sensor. For example, the multiconcentration multidot pattern 400C can be used to generate a calibration curve for an analyte based on measures at microdots 402A, 402B and 402C. Such calibration curve can be used to estimate a saturation point of the analyte for a given sensor. Moreover, the calibration curve can be used to predict sensor performance to a given concentration of analyte.

FIG. 4D is a diagram that illustrates a multianalyte pattern 400D, in accordance with examples. The multianalyte pattern 400D of FIG. 4D illustrates the use of microdots 402, 404 having different analytes. For example, microdot 402 may have had one particular analyte deposited while microdot 404 may contain a different analyte. The use of a multianalyte pattern 400D may enable multiple linear calibration curves to be generated for a given sensor for a variety of possible analytes that may be used in subsequent assays.

FIG. 4E is a diagram that illustrates a multidot multianalyte pattern 400E, in accordance with examples. The multidot multianalyte pattern 400E of FIG. 4E illustrates the use of a predetermined concentration of multiple analytes. For example, a predetermined concentration for each analyte may be used and multiple microdots deposited for each analyte. The use of multidot multianalyte pattern 400E may enable more accurate linear curves to be generated for a given sensor for a variety of possible analytes.

FIG. 4F is a diagram that illustrates a multiconcentration multianalyte pattern 400F, in accordance with examples. The multiconcentration multianalyte pattern 400F of FIG. 4F illustrates the use of multiple analytes having multiple microdots of different concentrations. The use of a multiconcentration multianalyte pattern 400F on a sensor may enable multiple calibration curves to be generated for a variety of possible analytes to be used in subsequent assays.

FIG. 4G is a diagram that illustrates an edge pattern 400G, in accordance with examples. The edge pattern 400G of FIG. 4G illustrates the use of multiple microdots 402 of a single analyte of predetermined concentration at the edges of a sensor surface. For example, the microdots 402 may be placed near the perimeter of the sensor and away from the center of the sensor. The use of an edge pattern 400G may free space in the center of the sensor, thus allowing for the center of the sensor to be sampled in an assay without interference from the microdots.

FIG. 4H is a diagram that illustrates a combination pattern 400H, in accordance with examples. The combination pattern 400H of FIG. 4H illustrates the use of any of the other patterns 400A-400G described above. The use of a combination pattern 400H may thus enable any of the benefits of the patterns 400A-400G above, as well as providing such benefits more efficiently by including all the microdots on the same sensor.

The block diagrams of FIGS. 4A-4H are not intended to indicate that the example patterns 400A-400H are to include all of the components shown in FIGS. 4A-4H. Further, the patterns 400A-400H may include any number of additional components not shown in FIGS. 4A-4H, depending on the details of the specific implementation. For example, the combination pattern 400H or other patterns 400A-400G may include additional microdots, analytes, or patterns.

FIGS. 5A-5D are diagrams illustrating various calibration regions that can be used for depositing microdots onto a sensor. FIG. 5A illustrates a separate region 500A for calibration, in accordance with examples. The separate region 500A of FIG. 5 illustrates a separate calibration region 502 coupled to a primary portion 504 of a surface-enhanced substrate. For example, the separate region may have a microdot 402 deposited thereon. The use of a separate calibration region 502 may enable subsequent assays to be performed on the primary portion 504 without any interference from the analyte in the microdot 402.

FIG. 5B illustrates multiple separate regions 500B for calibration, in accordance with examples. The multiple separate regions 500B illustrates two separate calibration regions 502 coupled to opposing sides of the primary portion 504. The use of multiple separate calibration regions 502 may enable an average reading to be taken of micro-assay measurements and thus generate a more accurate estimate of performance of the chip with respect to an analyte.

FIG. 5C illustrates a laser-treatable calibration region 500C. The laser-treatable calibration region 500C includes a treatable region 506 inside the primary portion 504 of the surface-enhanced substrate. For example, a degradable microdot 508 is deposited onto the treatable region 506 and a micro-assay is performed. The degradable microdot 508 includes an analyte that is a degradable molecule. The treatable region 506 may then be treated using a laser, or any other suitable method for removing the analyte. Thus, the analyte molecules can be removed from the treatable region 506 prior to performing a subsequent assay on the primary portion 504 of the surface-enhanced substrate.

FIG. 5D illustrates a detachable region 500D for calibration, in accordance with examples. The detachable region 500D includes a separate calibration region 502 that includes a microdot 402. As indicated by a dotted line 510, the separate calibration region 502 can be broken off and removed from the primary portion 504. Thus, the analyte molecules can be removed with the detachable separate calibration region 502 prior to performing a subsequent assay on the primary portion 504 of the surface-enhanced substrate.

The block diagrams of FIG. 5A-5D are not intended to indicate that the example calibration regions 500A-500D are to include all of the components shown in FIGS. 5A-5D. Further, the calibration regions 500A-500D may include any number of additional components not shown in FIGS. 5A-5D depending on the details of the specific implementation. For example, although a single analyte having a single predetermined concentration is shown, in some examples multiple analytes having a concentration may be used as described with respect to FIG. 4.

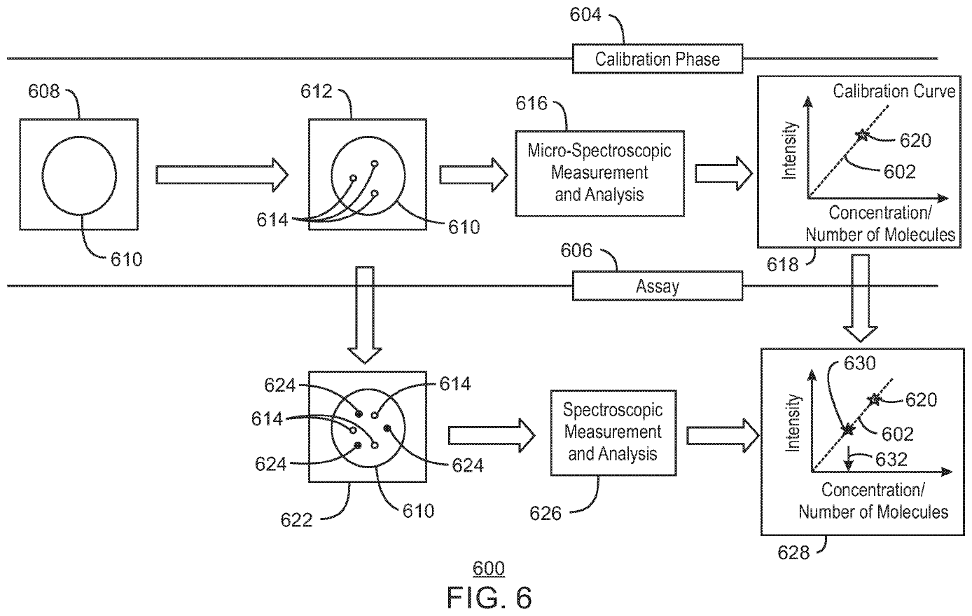

FIG. 6 is a schematic diagram illustrating an example method 600 for generating and using a calibration curve 602. The schematic diagram includes a calibration phase 604 and an assay 606. For example, the calibration curve 602 can be generated during the calibration phase 604 and used in the assay 606. For example, the assay 606 may be a SERS Assay, a SEIRA Assay, or a SEL assay.

As shown in FIG. 6, the method 600 includes, at block 608, receiving an analysis chip with a surface-enhanced substrate 610. For example, the surface-enhanced substrate may include a substrate with a surface enhancement, such as the nanopillars with respect to FIG. 3. The method 600 further includes, at block 612, depositing microdots 614 onto the surface-enhanced substrate 610. For example, the microdots 614 may include a predetermined concentration of an analyte. The method also includes, at block 614, performing a micro-spectroscopic measurement and analysis on the deposited microdots. For example, the surface-enhanced substrate 610 may be illuminated using any suitable light source, such as the light sources described in FIGS. 1 and 2.

The spectral content of emitted light from the surface-enhanced substrate 610 may then be measured and analyzed at block 616. In one example, the emitted light is measured using a Raman microscope. The Raman microscope used for a micro-Raman measurement may be a high spatial resolution microscope. In some examples, the analysis includes determining shifts in wavelengths as compared to the spectral content of the light from the light source. In some examples, the measured intensity of emitted light from the microdots 614 is averaged.

At block 618, a calibration curve 602 is generated based on the spectral content of the microdots. For example, given a particular averaged Raman intensity and the predetermined concentration of analyte in the microdots, a linear function may be generated through the origin of the axes and a point 620 indicating having one coordinate representing the average Raman-intensity of the microdots and another coordinate corresponding to the predetermined concentration of analyte.

At block 622, a target analyte 624 is dispensed onto the surface-enhanced substrate 610. In some examples, the target analyte 624 is deposited onto the surface-enhanced substrate 610 using a microfluidic ejector. For example, the target analyte 624 can be disposed using a thermal inkjet (TIJ) or a piezo inkjet (PIJ) printer. In some examples, the target analyte 624 is dispensed onto the surface-enhanced substrate 610 using any other suitable preparation method. In one example, the analysis chip is dipped or soaked in a solution containing the target analyte 624. In another example, the analysis chip is sprayed with a solution containing the target analyte 624. In another example, the analysis chip is exposed to a volatile compound including the analyte. For example, the volatile compound may be a solvent for the analyte. In some examples, the volatile compound includes an alcohol, such as methanol, a ketone, such as acetone, or any number of other materials.

The method 600 also further includes, at block 626, performing a spectroscopic measurement and analysis. For example, the spectroscopic measurement may be a Raman measurement or a Fourier transform infrared (FTIR) measurement, among other possible spectroscopic measurements. In some examples, intensity values for the microdots containing the target analyte 624 may be measured.

At block 628, a second calibration curve can be generated. The intensity values can be averaged and placed on a point 630 in the calibration curve 602 in order to generate a measurement readout 632 indicating a particular concentration or number of molecules of analyte associated with the intensity. Thus, the calibration curve 602 may be provided with the analysis chip to allow an unknown concentration of a target analyte 624 to be determined based on the calibration curve 602, along with an estimate of the measurement confidence.

It is to be understood that the process diagram of FIG. 6 is not intended to indicate that all of the elements of the method 600 are to be included in every case. Further, any number of additional elements not shown in FIG. 6 may be included in the method 600, depending on the details of the specific implementation.

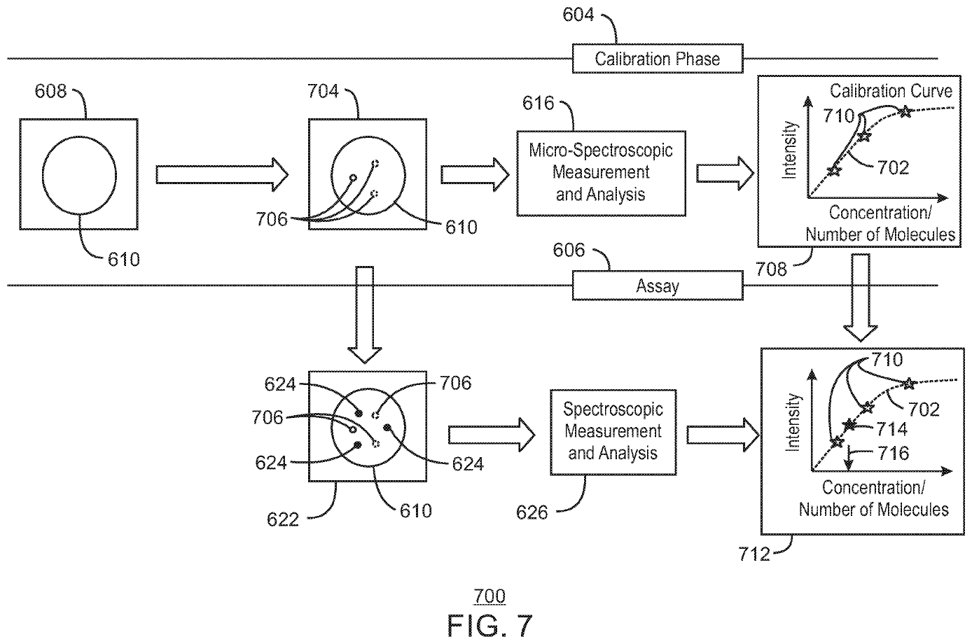

FIG. 7 is a schematic diagram illustrating an example method 700 for generating a calibration curve. The method 700 can be implemented in the controller 118 of the systems of FIGS. 1 and 2 or the controller 118 of FIG. 10. For example, the method may be implemented using processor 1002.

The method 700 includes similarly numbered elements from FIG. 6. In addition, the method 700 includes, at block 704, depositing a set of microdots 706 having different predetermined concentrations of an analyte onto a surface-enhanced substrate 610. After micro-spectroscopic measurement and analysis at block 614, the method 700 further includes, at block 708, generating a calibration curve 702. In some examples, the calibration curve is generated by fitting a linear or a non-linear model. For example, the model chosen may be based on the analysis of spectral content from the micro-spectroscopic measurements 710. Generation of the calibration curve enables improved sampling of the linear response region of the surface-enhanced substrate, and also estimation of the saturation point of the analyte for the surface-enhance substrate. In addition, the calibration curve enables prediction of sensor response to a given concentration or amount of analyte molecules.

The method 700 also further includes, at block 712, plotting a spectroscopic measurement value 714 within the calibration curve 702 and generating a measurement readout 716 based on an associated value from the calibration curve 702. For example, given a particular Raman intensity provided by the spectroscopic measurement and analysis, a particular concentration can be generated as a measurement readout 716. Thus, given an analyte with an unknown concentration, the calibration curve 702 and spectroscopic measurement and analysis 626 can be used to determine the concentration of the analyte. Moreover, sampling the calibration curve can give insight into the binding capacity of the substrate, which can improve prediction of sensor performance on unknown amounts of analyte. For example, the binding capacity of the surface-enhanced substrate may be affected by factors such as metal surface quality and impurity levels, among other factors. In the case of substrates relying on mechanical properties of the substrate, such as the deformation of pillars, the calibration curve may give insight into mechanical properties of the substrate that may have been affected by the manufacturing process or storage conditions.

It is to be understood that the process diagram of FIG. 7 is not intended to indicate that all of the elements of the method 700 are to be included in every case. Further, any number of additional elements not shown in FIG. 7 may be included in the method 700, depending on the details of the specific implementation. For example, additional analytes may be deposited onto the analytes chips and additional measurements may be performed.

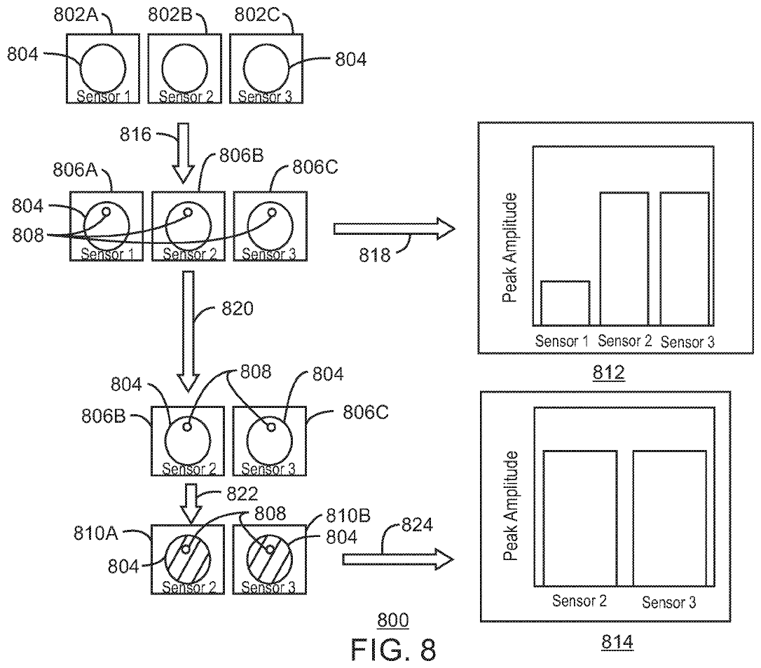

FIG. 8 is a schematic diagram illustrating an example method for performing a sensor performance assay to reject defective sensors. The method 800 of FIG. 8 can be implemented in the controller 118 of the systems of FIGS. 1 and 2 or the controller 118 of FIG. 10. For example, the method may be implemented using processor 1002.

The diagram of FIG. 8 includes a first set of sensors 802A, 802B, 802C with surface-enhanced substrates 804. FIG. 8 also includes a second set of sensors 806A, 806B, 806C with microdots 808 on the surface-enhanced substrates 804. FIG. 8 also further includes a third set of sensors 810A and 810B, on which an analyte has been introduced to the surface-enhanced substrates 804, as indicated by shading. For example, sensors 810A and 8108 may correspond to sensors 806B and 806C, respectively. FIG. 8 also includes a first graph 812 indicating peak amplitudes of the set of sensors 806A, 806B, 806C and a second graph 814 indicating peak amplitudes of the set of sensors 810A and 810B.

The method 800 of FIG. 8 includes depositing a microdot 808 including an analyte onto the surface-enhanced substrate 804 each of the sensors 802A, 802B, and 802C to generate sensors 806A, 806B, and 806C, as indicated by an arrow 816. The method 800 further includes, as indicated by an arrow 818, performing a micro-assay on the sensors 806A, 806B, and 806C to generate a first graph 812. As shown in the first graph 812, sensor 1 806A has a peak amplitude that is significantly less than other sensors 806B and 806C. Accordingly, sensor 1 806A is removed before further assays are performed. The method 800 thus includes filtering out, at arrow 820, the first sensor 806A and sending sensors 806B and 806C out for further analysis.

The method 800 includes dispensing an analyte onto the surface-enhanced substrate 804 of the sensors 806B and 806C to generate sensors 810A and 810B, as indicated by arrow 822. The method 800 includes performing an assay to generate a second graph 814, as shown by arrow 824. The second graph 814 shows that sensors 810A and 8108 have a similar peak amplitude. Thus, defective sensors can be filtered out 820 in advance based on the outcome of the micro-assay 818 before the sensors are delivered for additional performance testing.

It is to be understood that the process diagram of FIG. 8 is not intended to indicate that all of the elements of the method 800 are to be included in every case. Further, any number of additional elements not shown in FIG. 8 may be included in the method 800, depending on the details of the specific implementation. For example, additional analytes may be deposited onto the analytes chips and additional measurements may be performed. In addition, the microdots may be arranged in particular patterns and the surface-enhanced substrates may be arranged in various configurations as described in FIGS. 4 and 5.

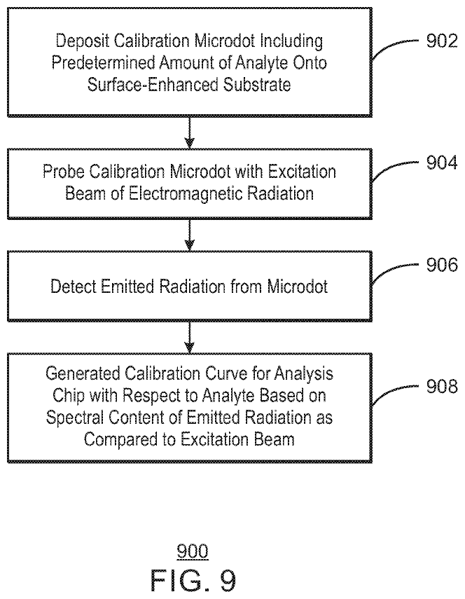

FIG. 9 is a process flow diagram illustrating an example method for filtering sensors based on an estimated performance. The method 900 of FIG. 9 can be implemented in the controller 118 of the systems of FIGS. 1 and 2 or the controller 118 of FIG. 10. For example, the method may be implemented using processor 1002.

At block 902, a microfluidic ejector deposits a microdot onto a surface-enhanced substrate of an analysis chip. In some examples, the microdot includes a predetermined concentration of an analyte. In some examples, the microdot may be one of a plurality of microdots having predetermined concentration of an analyte. In some examples, the microdot may be one of a plurality of microdots having different predetermined concentrations of an analyte.

At block 904, an optical system probes the microdot with an excitation beam of electromagnetic radiation. For example, the excitation beam may be generated by a source of electromagnetic radiation such as a light source.

At block 906, the processor detects emitted radiation from the microdot. For example, the emitted radiation may include light with shifted wavelengths as compared to the light from the light source.

At block 908, the processor generates a calibration curve for the analysis chip with respect to the analyte based on spectral content of the emitted radiation as compared to the excitation beam. In some examples, the calibration curve is a linear curve or a non-linear curve.

It is to be understood that the process diagram of FIG. 9 is not intended to indicate that all of the elements of the method 900 are to be included in every case. Further, any number of additional elements not shown in FIG. 9 may be included in the method 900, depending on the details of the specific implementation. For example, the method 900 may include dispensing a target analyte onto the analysis chip, performing a spectroscopic measurement of the target analyte, and determining a concentration of the target analyte by comparing the spectroscopic measurement to the calibration curve. In some examples, the method 900 may include estimating a saturation point of the analyte for the analysis chip based on the calibration curve. Furthermore, in some example, the method 900 may include sampling the calibration curve to estimate a binding capacity of the surface-enhanced substrate. In some examples, the method 900 may also further include laser treating the microdot. For example, the analyte may be a degradable molecule.

FIG. 10 is a drawing of a controller 118 to generate calibration curves and perform analysis of spectral content, in accordance with examples. The controller 118 includes a central processing unit (CPU) 1002 that executes stored instructions. In various examples, the CPU 1002 is a microprocessor, a system on a chip (SoC), a single core processor, a dual core processor, a multicore processor, a number of independent processors, a computing cluster, and the like.

The CPU 1002 is communicatively coupled to other devices in the controller 118 through a bus 1004. The bus 1004 may include a peripheral component interconnect (PCI) bus, and industry standard architecture (EISA) bus, a PCI express (PCIe) bus, high-performance interconnects, or a proprietary bus, such as used on a system on a chip (SoC).

The bus 1004 may couple the CPU 1002 to a graphics processing unit (GPU) 1006, such as units available from Nvidia, Intel, AMD, ATI, and others. If present, the GPU 1006 provides graphical processing capabilities to enable the high-speed processing of images from the camera. The GPU 1006 may be configured to perform any number of graphics operations. For example, the GPU 1006 may be configured to pre-process the plurality of image frames by isolating regions on which to print microdots, downscaling, reducing noise, correcting lighting, and the like. In examples that use only spectroscopic techniques, the GPU 1006 may not be present.

A memory device 1008 and a storage device 1010 may be coupled to the CPU 1002 through the bus 1004. In some examples, the memory device 1008 and the storage device 1010 are a single unit, e.g., with a contiguous address space accessible by the CPU 1002. The memory device 1008 holds operational code, data, settings, and other information used by the CPU 1002 for the control. In various embodiments, the memory device 1008 includes random access memory (RAM), such as static RAM (SRAM), dynamic RAM (DRAM), zero capacitor RAM, embedded DRAM (eDRAM), extended data out RAM (EDO RAM), double data rate RAM (DDR RAM), resistive RAM (RRAM), and parameter RAM (PRAM), among others.

The storage device 1010 is used to hold longer-term data, such as stored programs, an operating system, and other code blocks used to implement the functionality of the system. In various examples, the storage device 1010 includes non-volatile storage devices, such as a solid-state drive, a hard drive, a tape drive, an optical drive, a flash drive, an array of drives, or any combinations thereof. In some examples, the storage device 1010 includes non-volatile memory, such as non-volatile RAM (NVRAM), battery backed up DRAM, flash memory, and the like. In some examples, the storage device 1010 includes read only memory (ROM), such as mask ROM, programmable ROM (PROM), erasable programmable ROM (EPROM), and electrically erasable programmable ROM (EEPROM).

A number of interface devices may be coupled to the CPU 1002 through the bus 1004. In various examples, the interface devices include a microfluidic ejector controller (MEC) interface 1012, an imager interface 1016, and a motor controller 1020, among others.

The MEC interface 1012 couples the controller 118 to a microfluidic ejector controller 1014. The MEC interface 1012 directs the microfluidic ejector controller 1014 to fire microfluidic ejectors in a microfluidic ejector array, either individually or as a group. As described herein, the firing may be performed during imaging of a particular region of a microfluidic ejector array.

The imager interface 1016 couples the controller 118 to an imager 1018. The imager interface 1016 may be a high-speed serial or parallel interface, such as a PCIe interface, a USB 3.0 interface, a FireWire interface, and the like. In various examples, the imager 1018 is a high frame-rate camera configured to transfer data and receive control signals over the high-speed interface. In some examples, the imager 1018 is a multichannel spectroscopic system, or other optical device.

The motor controller 1020 couples the controller 118 to a stage translator 1022. The motor controller 1020 may be a stepper motor controller or a servo motor controller, among others. The stage translator 1022 includes a motor, a sensor, or both, coupled to the motor controller 1020 to move the stage and attached print medium or collection vessels, under a microfluidic ejector.

A network interface controller (NIC) 1024 may be used to couple the controller 118 to a network 1026. In various examples, this allows for the transfer of control information to the controller 118 and data from the controller 118 to units on the network 1026. The network 1026 may be a wide area network (WAN), a local area network (LAN), or the Internet, among others. In some examples, the NIC 1024 connects the controller 118 to a cluster computing network, or other high-speed processing system, where image processing and data storage occur. This may be used by controllers 118 that do not include a GPU 1006 for graphical processing. In some examples, a dedicated human machine interface (HMI) (not shown) may be included in the controller 118 for local control of the systems. The HMI may include a display and keyboard.

The storage device 1010 may include code blocks used to implement the functionality of the system. In various examples, the code blocks include a capture controller 1028 that is used to capture images from the imager 1018. For example, the images may depict surface-enhanced substrates having a microdot. In some examples, a GPU 1006 is used to identify a region including a surface-enhanced substrate and process the region to detect locations in which to deposit microdots or to detect spectral content from a microdot in the region.

An image processor 1030 processes captured images to detect spectral content. In various examples, the spectral content includes an intensity level of a particular portion of the spectrum from one of more of the microdots.

A stage motion controller 1032 directs the motor controller 1020 to move the stage translator 1022. In some examples, the motor controller 1020 is used to move a deposit medium, such as an analysis chip including a surface-enhanced substrate, under a microfluidic ejector array. In other examples, the motor controller 1020 is used to move an analysis chip including a deposited microdot into a light source for imaging by the imager 1018.

An MEC firing controller 1034 uses the MEC interface 1012 to direct a microfluidic ejector controller 1014 to fire a microfluidic ejector. In some examples, this is performed to deposit a microdot including an analyte onto a surface-enhanced substrate of an analysis chip for micro-assay analysis. In other examples, this is performed to deposit microdots or any other pattern of analytes onto surface-enhanced substrate of an analysis chip for assay analysis.

A calibration curve generator 1036 uses images from the image 1018 to extract spectral content associated with a microdot or other pattern associated with an analyte. In some examples, the calibration curve generator 1036 calculates a calibration curve based on spectral content associated with the analyte. For example, the calibration curve can be linear or non-linear based on the spectral content. In some examples, the calibration curve generator 1036 generates a calibration curve based on spectral content from microdots having different concentrations of an analyte.

Although shown as contiguous blocks, the logic components may be stored in any order or configuration. For example, if the storage is a hard drive, the logic components may be stored in non-contiguous, or even overlapping, sectors.

While the present techniques may be susceptible to various modifications and alternative forms, the examples discussed above have been shown only by way of example. It is to be understood that the technique is not intended to be limited to the particular examples disclosed herein. Indeed, the present techniques include all alternatives, modifications, and equivalents falling within the true spirit and scope of the appended claims.

What is claimed is:

1. A method comprising:
depositing a microdot onto a surface-enhanced substrate of an analysis chip, the microdot comprising a predetermined concentration of an analyte;
probing the microdot with an excitation beam of electromagnetic radiation;
detecting emitted radiation from the microdot; and
generating a calibration curve for the analysis chip with respect to the analyte based on spectral content of the emitted radiation as compared to the excitation beam.

2. The method of claim 1, comprising dispensing a target analyte onto the analysis chip, performing a spectroscopic measurement of the target analyte, and determining a concentration of the target analyte by comparing the spectroscopic measurement to the calibration curve.

3. The method of claim 1, comprising depositing a plurality of microdots with different predetermined concentrations of the analyte, and estimating a saturation point of the analyte for the analysis chip based on the calibration curve.

4. The method of claim 1, comprising depositing a plurality of microdots with different predetermined concentrations of the analyte, wherein sampling the calibration curve to estimate a binding capacity of the surface-enhanced substrate.

5. The method of claim 1, comprising laser treating the microdot, wherein the analyte comprises a degradable molecule.

6. An apparatus comprising, a surface-enhanced substrate having a microdot deposited onto a surface thereon via a microfluidic ejector, the microdot comprising a predetermined concentration of an analyte.

7. The apparatus of claim 6, wherein the microdot is deposited in a predetermined calibration region of the surface-enhanced substrate.

8. The apparatus of claim 6, wherein the surface-enhanced substrate comprises a plurality of microdots having the predetermined concentration of the analyte.

9. The apparatus of claim 6, wherein the surface-enhanced substrate comprises a plurality of microdots having different predetermined concentrations of the analyte.

10. The apparatus of claim 6, wherein the microdot is deposited along an edge of the surface-enhanced substrate.

11. The apparatus of claim 6, wherein the surface-enhanced substrate comprises a plurality of microdots having different predetermined concentrations of a plurality of analytes.

12. The apparatus of claim 6, wherein the microdot is deposited in a predetermined calibration region of the surface-enhanced substrate comprising a separate calibration surface coupled to a primary portion of the surface-enhanced substrate.

13. The apparatus of claim 6, wherein the microdot is deposited in a plurality of predetermined calibration regions of the surface-enhanced substrate comprising separate calibration surfaces coupled to a primary portion of the surface-enhanced substrate.

14. The apparatus of claim 6, wherein the microdot is deposited in a predetermined calibration region of the surface-enhanced substrate comprising a breakable separate calibration surface coupled to a primary portion of the surface-enhanced substrate.

15. The apparatus of claim 6, wherein the analyte comprises a degradable molecule to be laser treated.
